(19) United States
(12) Patent Application Publication
(10) Pub. No.: US 2026/0360582 A1
(43) Pub. Date: Nov. 12, 2026

(54) IMPROVED AND SAFE TECHNIQUES FOR USE OF RADIOACTIVE GENERATOR

(71) Applicant: Jubilant Draximage Inc., Montreal (CA)

(72) Inventors: Cristian Leonardo Juverdianu, Montreal (CA); Anita Jolene MacDonald, Montreal (CA); Abmel Xiques Castillo, Montreal (CA); Indranil Nandi, Yardley, PA (US)

(73) Assignee: Jubilant Draximage Inc., Montreal, QC (CA)

(21) Appl. No.: 19/132,956

(22) PCT Filed: Nov. 30, 2023
(86) PCT No.: PCT/IB2023/062060

§ 371 Date: May 27, 2025

Related U.S. Application Data
(60) Provisional application No. 63/385,580, filed on Nov. 30, 2022.

(51) Int. Cl. G21G 1/00; A61B 6/03

(52) U.S. Cl. CPC G21G 1/00; A61B 6/037

(57) ABSTRACT

The present invention provides a radioisotope elution system for an improved method for the quality control testing of a radioactive generator. In particular, the present invention relates to a radioisotope elution system and its improved method for quality control testing in order to identify and/or extend the shelf-life of the radioactive generator, wherein the said radioisotope elution system is able to produce an increased amount of radioactive eluate by initiating two or more consecutive elutions at an increased flow rate and/or increased volume of the eluant; and wherein the shelf-life of the radioisotope generator is detected by measuring the enhancement of signal-to-noise ratio towards the end of the generator life.

Radiopharmaceuticals are important for therapeutic and diagnostic use of various diseases. The safe and efficient use of these important and dangerous radiopharmaceutical isotopes having either short or long half-life are essential during their intended use by patient and/or healthcare professionals. Most of the widely accessible radiopharmaceuticals are generated by various known techniques and are supplied as radioisotope generators comprising a column containing the parent long half-life isotope. Some desired isotopes like Rb-82 used for PET imaging are produced in-situ through an elution process wherein long half-life undesirable radioisotopes like Sr-82 and Sr-85 are also present. These undesirable radioisotopes can cause adverse and harmful side effects inside the patient's body after administration. Therefore, FDA has mentioned a certain amount of total volume of eluate and stated that after 28-42 days there is a chance of expiration of the generator, which is an important aspect because the radioisotopes having long half-lives can cause severe harmful damage if infused into the patient's body, more than the standard limits. In radiopharmaceutical art, there is still an unmet need to ensure safe and efficient use of daughter radioisotope near to the end-life of generator.

### SUMMARY

The present invention relates to a radioisotope elution system for quality control testing of a radioactive generator. In particular, the present invention relates to a radioisotope elution system and an improved method for quality control testing in order to identify and/or extend the shelf-life of the radioactive generator.

It is an object of the present invention to provide an extended shelf-life of the radioisotope generator of a radioisotope elution system, wherein the said radioisotope elution system is able to produce an increased amount of radioactive eluate by initiating two or more consecutive elutions at an increased flow rate and/or an increased volume of the eluant.

It is an object of the present invention to determine the shelf-life of the radioisotope generator of a radioisotope elution system, wherein the shelf-life of the said radioisotope generator is measured by the enhancement of signal-to-noise ratio.

It is an object of the present invention to provide extended life of the radioisotope generator of a radioisotope elution system, wherein the shelf-life of the said radioisotope generator is measured by the enhancement of signal-to-noise ratio.

It is an object of the present invention to generate an increase of radioactivity of the daughter radioisotope eluted from the parent radioisotope within the radioisotope generator by commencing two or more consecutive elutions.

It is an object of the present invention to provide an extended shelf-life of the radioisotope generator of a radioisotope elution system, wherein the said radioisotope elution system is able to produce an increase of radioactivity of radioactive eluate by initiating two or more consecutive elutions at an increased flow rate and also perform back to back daily quality controls (DQCs) testing.

It is an object of the present invention to generate an increase of radioactivity of daughter radioisotope eluted from parent radioisotope within the radioisotope generator and/or determine the shelf-life of the radioactive generator by the enhancement of signal-to-noise ratio.

It is an object of the present invention to determine and/or identify the shelf-life of the radioisotope generator by measuring the enhancement of coefficient of variation of signal-to-noise ratio and/or contrast to noise ratio based on the consecutive elutions, wherein the extended shelf-life of the said radioactive generator is about 60 days or more.

It is an object of the present invention to provide a method of diagnosing and/or treating cardiovascular events and/or diseases in a subject. Further, it is an object of the present invention to provide a method of diagnosing and/or treating cardiovascular diseases selected from the group consisting of peripheral arterial disease (PAD), coronary artery disease (CAD), cardiovascular stroke, myocardial ischemic, renal perfusion, brain perfusion, microvascular disease, ischemic heart attack, myocardial blood flow (MBF), or the like in a subject.

It is an object of the present invention to provide a radioisotope elution system comprising:
a) a radioisotope generator to generate a daughter radioisotope eluting from the parent radioisotope by using a suitable eluant;
b) a detector to determine the radioactivity of the daughter radioisotope;
c) a generator line that permits fluid communication between the reservoir to the generator; and
d) a memory communicatively coupled to the processor when the system is operational, the memory bearing processor-executable instructions when executed on the processor;
   wherein the said radioisotope elution system is able to produce an increased volume of radioactive eluate by initiating two or more consecutive elutions at an increased flow rate, and wherein the shelf-life of radioisotope generator is detected by measuring the enhancement of signal-to-noise ratio towards the end of a generator life.

The present invention includes the elution system, wherein the radioisotope generator can be selected from ⁸²Sr/⁸²Rb, ¹⁰⁹Cd/^{109m}Ag, ¹¹⁸Te/¹¹⁸Sb, ¹³⁷Cs/^{137m}Ba, ¹³⁴Ce/¹³⁴La, ¹⁴⁴Ce/¹⁴⁴Pr, ¹⁴⁰Nd/¹⁴⁰Pr, ¹⁶⁷Tm/^{167m}Er, ¹⁷⁸W/¹⁷⁸Ta, ¹⁹¹Os/^{191m}Ir, and thereof.

The present invention includes the radioisotope elution system, wherein the radioisotope generator is ⁸²Sr/⁸²Rb.

An embodiment of the present invention includes the elution system, wherein the ⁸²Sr/⁸²Rb radioisotope generator comprises a semi-automated infusion system.

An embodiment of the present invention includes the elution system, wherein the ⁹⁹Mo/^{99m}Tc radioisotope generator comprises a semi-automated infusion system.

An embodiment of the present invention includes the radioisotope elution system, wherein the detector is selected from the group consisting of positron detector, PMT detector, CZT detector, or combinations thereof.

An embodiment of the present invention includes the radioisotope elution system, wherein the increased volume and/or flow rate is in order to increase the amount of daughter radioisotope.

An embodiment of the present invention includes the radioisotope elution system, wherein two or more consecutive elutions are able to produce an increased amount of radioactive eluate.

An embodiment of the present invention includes the radioisotope elution system, wherein the amount of radioactive eluate is increased from about 13% to about 120%.

An embodiment of the present invention includes the radioisotope elution system, wherein back-to-back daily quality control testing is performed to identify the purity of the daughter radioisotope.

An embodiment of the present invention includes the radioisotope elution system, wherein the shelf-life of the radioactive generator is determined by the enhancement of signal-to-noise ratio.

An embodiment of the present invention includes a radioisotope elution system comprising:
a) a radioisotope generator to generate a daughter radioisotope eluting from the parent radioisotope by using a suitable eluant;
b) a detector to determine the radioactivity of the daughter radioisotope;
c) a generator line that permits fluid communication between the reservoir to the generator;
d) a memory communicatively coupled to the processor when the system is operational, the memory bearing processor-executable instructions when executed on the processor;
e) a controller; and
f) a user interface;
   wherein the said radioisotope generator is an ⁸²Sr/⁸²Rb generator and the shelf-life of radioisotope generator is detected by measuring the enhancement of signal-to-noise ratio towards the end of a generator life, and wherein the said radioisotope elution system is able to produce an increased volume of radioactive eluate by initiating two or more consecutive elutions at an increased flow rate.

An another embodiment of the present invention includes the radioisotope elution system, wherein the threshold is an ⁸²Sr activity less than 0.02 μCi.

An embodiment of the present invention includes a radioisotope elution system comprising:
a) a radioisotope generator to generate a daughter radioisotope eluting from the parent radioisotope by using a suitable eluant;
b) a detector to determine the radioactivity of the daughter radioisotope;
c) a generator line that permits fluid communication between the reservoir to the generator;
d) a memory communicatively coupled to the processor when the system is operational, the memory bearing processor-executable instructions when executed on the processor;
e) a controller; and
f) a user interface;
wherein the said radioisotope generator can be selected from the group consisting of ⁹⁹Mo/^{99m}Tc, ⁹⁰Sr/⁹⁰Y, ⁸²Sr/⁸²Rb, ¹⁸⁸W/¹⁸⁸Re, ⁶⁸Ge/⁶⁸Ga, ⁴²Ar/⁴²K, ⁴⁴Ti/⁴⁴Sc, ⁵²Fe/^{52m}Mn, ⁷²Se/⁷²As, ⁸³Rb/^{83m}Kr, ¹⁰³Pd/^{103m}Rh, ¹⁰⁹Cd/^{109m}Ag, ¹¹³Sn/^{113m}In, ¹¹⁸Te/¹¹⁸Sb, ¹³²Te/¹³²I, ¹³⁷Cs/^{137m}Ba, ¹⁴⁰Ba/¹⁴⁰La, ¹³⁴Ce/¹³⁴La, ¹⁴⁴Ce/¹⁴⁴Pr, ¹⁴⁰Nd/¹⁴⁰Pr, ¹⁶⁶Dy/¹⁶⁶Ho, ¹⁶⁷Tm/^{167m}Er, ¹⁷²Hf/¹⁷²Lu, ¹⁷⁸W/¹⁷⁸Ta, ¹⁹¹Os/^{191m}Ir, ¹⁹⁴⁰) s/¹⁹⁴Ir, ²²⁶Ra/²²²Rn, and ²²⁵Ac/²¹³Bi generators, and thereof; wherein the shelf-life of radioisotope generator is detected by measuring the enhancement of signal-to-noise ratio towards the end of a generator life, and wherein the said radioisotope elution system is able to produce an increased volume of radioactive eluate by initiating two or more consecutive elutions at an increased flow rate.

Currently available generator in the United States, such as the RUBY-FILL® generator (⁸²Sr/⁸²Rb generator) manufactured by Jubilant Draximage, Inc. is approved for 60 days post-calibration duration. Therefore, towards the end-life of the generator, the detection accuracy decreases. The inventors of the present invention developed an improved method of quality control tests to ensure excellent accuracy for the detection of Strontium if the generator expiry date gets extended at some point. Currently, as the generators are loaded with 100mCi of Sr-82, there remains >20mCi of Sr-82 (or >20mCi of Rb-82) at the end of life of the radioisotope generator. Since there could be an indication for pediatric use, which are in small doses, and there continues to be an increase in the number of solid state/digital cameras being used in clinics. It is possible to identify the smaller doses as required for pediatric or light-weight patients with the said newer digital camera systems. There is a need to identify and/or extend the life of the radioisotope generator for clinical utility.

### DETAILED DESCRIPTION

The present invention relates to a radioisotope elution system for quality control testing of a radioactive generator. In particular, the present invention relates to a radioisotope elution system and an improved method for quality control testing in order to identify and/or extend the shelf-life of the radioactive generator.

The present invention relates to a radioisotope elution system and method to provide extended shelf-life of the radioisotope generator, wherein the said radioisotope elution system is able to produce an increased volume of radioactive eluate by initiating two or more consecutive elutions at an increased flow rate.

The present invention further provides an improved method to accurately and quantitatively identify the shelf-life of the radioisotope generator by the enhancement of signal-to-noise ratio based on two or more consecutive elutions.

The present invention provides an improved method to accurately and quantitatively identify the shelf life of the radioisotope generator by the enhancement of the coefficient of variation of signal-to-noise ratio and/or contrast-to-noise ratio based on two or more consecutive elutions.

The present invention relates to a radioisotope elution system and its improved method for quality control testing in order to identify and/or extend the shelf-life of the radioactive generator, wherein the said radioisotope elution system is able to produce an increased amount of radioactive eluate by commencing two or more consecutive elutions at an increased flow rate, and wherein the shelf-life of the radioisotope generator is detected by measuring the enhancement of signal-to-noise ratio towards the end of the generator life.

The present invention relates to a radioisotope elution system and its improved method for quality control testing in order to identify and/or extend the shelf-life of the radioactive generator, wherein the said radioisotope elution system is able to produce an increased amount of radioactive eluate by commencing two or more consecutive elutions at an increased flow rate and/or increased volume of eluant, and wherein the shelf-life of the radioisotope generator is detected by measuring the enhancement of signal-to-noise ratio towards the end of the generator life.

The present invention can be more readily understood by reading the following detailed description of the invention and included embodiments.

As used herein, the term “imaging” refers to techniques and processes used to create images of various parts of the human body for diagnostic and treatment purposes within digital health. X-ray radiography, Fluoroscopy, Magnetic resonance imaging (MRI), Computed Tomography (CT), Medical Ultrasonography or Ultrasound Endoscopy Elastography, Tactile imaging, Thermography Medical photography, and nuclear medicine functional imaging techniques e.g. Positron Emission Tomography (PET), Dynamic Positron Emission Tomography or Single-photon Emission Computed Tomography (SPECT). Imaging seeks to reveal internal structures of the body, as well as to diagnose and treat disease.

As used herein, the term “Positron Emission Tomography (PET)” refers to a functional imaging technique that uses radioactive substances known as radiotracers or radionuclides to visualize and measure changes in metabolic processes, and in other physiological activities including blood flow, regional chemical composition, and absorption. Different tracers can be used for various imaging purposes, depending on the target process within the body commonly used radionuclide isotopes for PET imaging include carbon-11 (C-11), nitrogen-13 (N-13), oxygen-15 (O-15), fluorine-18 (F-18), rubidium-82 (Rb-82), copper-64 (Cu-64), zirconium-89 (Zr-89), and gallium-68 (Ga-68). The preferred radionuclide comprises Rb-82 (Rubidium-82) having a half-life of 75 seconds.

As used herein, the term “Single-Photon Emission Computed Tomography (SPECT)” refers to a nuclear medicine tomographic imaging technique using gamma rays and provide true 3D information. This information is typically presented as cross-sectional slices through the patient but can be freely reformatted or manipulated as required. The technique requires the delivery of a gamma-emitting radioisotope (a radionuclide) into the patient, normally through injection into the bloodstream. A marker radioisotope is generally attached to a specific ligand to create a radioligand, whose properties bind it to certain types of tissues. This allows the combination of ligand and radiopharmaceutical to be carried and bound to a region of interest in the body, where the ligand concentration is assessed by a gamma camera. SPECT agents include, iodine-123 (I-123), indium-111 (In-111), technetium-99m (Tc-99m), xenon-133 (Xe-133), thallium-201 (Tl-201), krypton-87m (Kr-81m), and gallium-67 (Ga-67). As used herein, the term “Computed Tomography (CT)” refers to computerized x-ray imaging in which a beam of x-rays aimed at a patient and rotated around the body, producing signals that are processed by the machine's computer to generate cross-sectional images of the body.

These slices are called tomographic images and contain more detailed information than conventional x-rays. Once the machine's computer collects a number of successive slices, they can be digitally “stacked” together to form a three-dimensional image of the patient that allows for easier identification and location of basic structures as well as possible tumors or abnormalities.

As used herein, the term “Magnetic Resonance Imaging (MRI)” is a non-invasive imaging technology that produces 3D detailed anatomical images, which is used for disease detection, diagnosis, and treatment monitoring. MRI is based on technology that excites and detects the change in the direction of the rotational axis of protons found in the water that makes up living tissues.

As used herein, the term “diagnosis” refers to a process of identifying a disease, condition, or injury from its signs and symptoms. A health history, physical exam, and tests, such as blood tests, imaging, scanning, and biopsies can be used to help make a diagnosis.

As used herein, the term “assessment” refers to a qualitative and/or quantitative assessment of the the myocardial blood flow and/or myocardial flow reserve, other diseases or abnormalities in a body part or region of interest (ROI).

As used herein, the term “automated infusion system” or “semi-automated infusion system” or “radionuclide generation” and/or “infusion system” or “Rb-82 elution system” refers to a system for generation and/or infusion of a radionuclide or radiotracer and administration into a subject. The automated infusion system or semi-automated infusion system comprises radioisotope generator, dose calibrator, computer, controller, display device, activity detector, cabinet, cart, waste bottle, sensors, shielding assembly, alarms or alerts mechanism, tubing, source vial, diluent or eluent, pump, and valves. The automated infusion system or semi-automated infusion system can be communicatively or electronically coupled to the imaging system.

As used herein, the term “therapy” or “therapeutic use” refers to the attempt to cure, improve, mitigate, treat and/or prevent disease and/or other conditions in humans. The term “therapy” also refers to pharmacotherapy or pharmacological therapy, which refers to the treatment of disease through the application of medications (drugs). It can be used to treat or prevent development of a disease, as well as to alleviate the pain and symptoms of the particular condition. Nuclear medicine therapy can be given with the help of radioisotopes like alpha emitters actinium-225 (Ac-225), astatine-211 (At-211), etc. and beta emitters such as lutetium-177 (Lu-177), lead-212 (Pb-212), etc.

As used herein, the term “Hybrid Molecular Imaging” refers to the fusion of two or more imaging technologies into a single, new form of imaging. This form is synergistic, which is more powerful than the sum of its parts. Hybrid imaging denotes image acquisitions on systems that ohysically combine complementary imaging modalities for an improved diagnostic accuracy and confidence as well as for imcreased for increased patient comfort. The hybrid imaging combines the strengths of two imaging modalities in one imaging session to more accurately diagnose and locate diseases while increasing patient comfort. These are generated by superimposing two images at two different spatial scales: the low-spatial scale is obtained by filtering one image with a low-pass filter; the high spatial scale is obtained by filtering a second image with a high-pass filter. Examples of hybrid imaging modalities include PET-CT, SPECT-CT and PET-MRI.

As used herein, the term “dose” refers to the dose of a radionuclide required to perform imaging in a subject. The dose of a radionuclide to be administered to the subject ranges from 0.27 μCi to 270 mCi.

As used herein, the term “coronary artery disease” or “cardiovascular disease” refers to a disease of major blood vessels. Cholesterol-containing deposits (plaques) in coronary arteries and inflammation are causes of coronary artery disease. The coronary arteries supply blood, oxygen, and nutrients to your heart. A buildup of plaque can narrow these arteries, decreasing blood flow to your heart. Eventually, the reduced blood flow may cause chest pain (angina), shortness of breath, or other coronary artery disease signs and symptoms. Significant blockage of the arteries can cause a heart attack. It can be diagnosed by imaging of the myocardium and/or myocardial blood flow (MBF) under rest or pharmacologic stress conditions to evaluate regional myocardial perfusion.

As used herein, the term “radionuclide” or “radioisotope” refers to an unstable form of a chemical element that releases radiation as it breaks down and becomes more stable. Radionuclides can occur in nature or can be generated in a laboratory. In medicine, they are used in imaging tests and/or in treatment.

As used herein, the term “radioisotope elution system” or “radioactive elution system” refers to an infusion system meant for generating a solution containing daughter radioisotope (eluate), measuring the radioactivity in the solution, and infusing the solution into a subject to perform various studies on the subject region of interest.

As used herein, the term “Sr/Rb elution system” or “⁸²Sr/⁸²Rb elution system” refers to an infusion system meant for generating a solution containing Rb-82, measuring the radioactivity in the solution, and infusing the solution into a subject to perform various studies on the subject region of interest.

As used herein, the term “image counts” refers to the number of radioisotope disintegrations acquired per unit of time by the PET scanner.

As used herein, the term “generator” or “radioisotope generator” refers to a hollow column inside a radio-shielded container. The column is filled with an ion exchange resin and radioisotope is loaded onto the resin. Radionuclide generator according to the present invention is selected from ⁹⁹Mo/^{99m}Tc, ⁹⁰Sr/⁹⁰Y, ⁸²Sr/⁸²Rb, ¹⁸⁸W/¹⁸⁸Re, ⁶⁸Ge/⁶⁸Ga, ⁴²Ar/⁴²K, ⁴⁴Ti/⁴⁴Sc, ⁵²Fe/^{52m}Mn, ⁷²Se/⁷²As, ⁸³Rb/^{83m}Kr, ¹⁰³Pd/^{103m}Rh, ¹⁰⁹Cd/^{109m}Ag, ¹¹³Sn/^{113m}In, ¹¹⁸Te/¹¹⁸Sb, ¹³²Te/¹³²I, ¹³⁷Cs/^{137m}Ba, ¹⁴⁰Ba/¹⁴⁰La, ¹³⁴Ce/¹³⁴La, ¹⁴⁴Ce/¹⁴⁴Pr, ¹⁴⁰Nd/¹⁴⁰Pr, ¹⁶⁶Dy/¹⁶⁶Ho, ¹⁶⁷Tm/^{167m}Er, ¹⁷²Hf/¹⁷²Lu, ¹⁷⁸W/¹⁷⁸Ta, ¹⁹¹Os/^{191m}Ir, ¹⁹⁴Os/¹⁹⁴Tr, ²²⁶Ra/²²²Rn, and ²²⁵Ac/²¹³Bi generators.

As used herein, the term “eluant” refers to the liquid or the fluid used for selectively leaching out the daughter radioisotopes from the generator column.

As used herein, the term “eluate” refers to the radioactive eluant after the acquisition of the daughter radioisotope from the generator column.

As used herein, the term “increased flow rate and/or increased volume” refers that the elution process comprises the step by initiating two or more consecutive elutions at an increased flow rate and/or increased volume of the eluant, wherein the flow rate of the eluant may vary from about 10 ml/min to about 100 ml/min in order to increase the amount of radioactive eluate. The consecutive elutions refer to at least two back-to-back elutions by which the amount and/or volume of the radioactive eluate may increase from about 13% to about 120%.

As used herein, the term “breakthrough” refers to the undesired elution of the parent radioisotope (i.e. above approved labeling limits) and/or impurities (i.e. Sr-85) in the eluate when the elution process occurs due to chemical and radiological decomposition or mechanical disruption of the stationary phase

As used herein, the term “controller” refers to a computer or a part thereof programmed to perform certain calculations, execute instructions, and control various activities of an elution system based on user input or automatically. The “controller” as referred to herein can control the activity detector, and one or more valves to control the fluid communication, user interface, alarm and/or sensor, or the like.

As used herein, the term “activity detector” refers to a component that is used to determine the amount of radioactivity present in eluate from a generator, e.g., prior to the administration of the eluate to the patient. The detector can detect the decay modes of Rb-82 by positron emission with a maximal energy of 3.15 MeV in 95.5% of decays or by electronic capture in 4.5% of decays with subsequent gamma emission.

As used herein, the term “alarm and/or sensor” refers to a system that is designed to alert an individual or group of individuals or a user at a specified time about the different parameters of the elution system and process. The sensor and/or alarm can sense an amount of eluate that has been dispensed from the generator. The sensor and/or alarm can be visible and/or audible and at least one of the visually and/or audibly perceptible exteriors of the elution system and produce light and sound to indicate to the user about the status of the parameters of the elution system, whether acceptable or unacceptable. The sensor and/or alarm automatically alarm the user about the limit of low radioactivity and a dynamic ability to calculate the signal-to-noise ratio and perform the back-to-back elutions in order to discriminate desired and undesired radioisotopes.

As used herein, the term “SNR” refers to signal-to-noise ratio, which is a measure of the end-life or shelf-life of the radioisotope generator. SNR or signal-to-noise ratio can be defined as a ratio of the target signal strength to the noise signal strength. End-life or shelf-life of the radioisotope generator is measured using signal-to-noise ratio by measuring the volume (yield) and/or flow rate of the radioisotope eluate during consecutive elutions, by performing back to back daily quality control testing and/or combinations thereof.

As used herein, the term “CNR” refers to the contrast-to-noise ratio, which is also a measure of the end-life or shelf-life of the radioisotope generator. CNR can be defined as a difference of target signal strength minus the background signal strength, divided by the noise signal strength.

As used herein, the term “image counts” refers to the number of radioisotope disintegrations acquired per unit of time by the PET scanner.

As used herein, the term “COV” refers to the coefficient of variance/variation, which is a measure of background noise signal to define the end-life or shelf-life of the radioisotope generator. The value of calculated COV is used for the calculation of SNR and CNR. As used herein, the term “myocardial blood flow (MBF)” can be defined as the volume of blood transiting through tissue at a certain rate. MFR (Myocardial Flow Reserve) constitutes the ratio of MBF during maximal coronary vasodilatation to resting MBF and is therefore impacted by both rest and stress flow. MFR represents the relative reserve of the coronary circulation.

As used herein, the term “about” refers to a measurable value such as a parameter, an amount, a temporal duration, and the like, and is meant to encompass variations of and from the specified value, in particular variations of +10% or less, preferably +5% or less from the specified value, such variations are appropriate to perform in the disclosed invention. It is to be understood that the value to which the modifier “about” refers is itself also specifically, and preferably, disclosed.

As used in the specification of the present invention, the singular forms “a”, “an”, and “the” include plural references unless the context clearly dictates otherwise. Thus, for example, a reference to “a process” or “a composition” includes one or more process or composition, with one or more steps or elements of the type described herein and/or which will become apparent to those persons skilled in the art upon reading this disclosure and so forth.

An embodiment of the present invention provides a method of diagnosing cardiovascular diseases in a subject comprising performing a PET scan, PET/CT scan, dynamic PET scan, SPECT scan, PET/MRI scan, MRI scan, or combinations thereof by administering a PET agent, a SPECT agent, a contrast agent, and a dye or combinations thereof.

In another embodiment, the present invention provides imaging protocols for diagnosing cardiovascular diseases in a subject. Imaging protocols are based on PET, SPECT CT, MRI, or combinations thereof.

In another embodiment, the present invention provides the radionuclide or radioisotope is selected from PET or SPECT agent. The PET or SPECT agent can be radiolabeled with one or more ligands or can be administered without radiolabeling. Another embodiment, the radionuclide or radioisotope is attached to the ligand before administration into the subject. The ligands are provided in a suitable dosage form and a radionuclide or radioisotope is attached to the ligand and then administered into the subject for imaging. The ligands according to the present invention are selected from but are not limited to Tetrofosmin, Sestamibi, and Fluorodeoxyglucose, or the like.

An embodiment, PET agents can be selected from carbon-11 (C-11), nitrogen-13 (N-13), oxygen-15 (O-15), fluorine-18 (F-18), rubidium-82 (Rb-82), copper-64 (Cu-64), zirconium-89 (Zr-89), and gallium-68 (Ga-68).

An embodiment, SPECT agents can be selected from iodine-123 (I-123), indium-111 (In-111), technetium-99m (Tc-99m), xenon-133 (Xe-133), thallium-201 (Tl-201), krypton-87m (Kr-81m), and gallium-67 (Ga-67).

An embodiment of the present invention includes a radioisotope elution system comprising:
a) a radioisotope generator to generate a daughter radioisotope eluting from the parent radioisotope by using a suitable eluant;
b) a detector to determine the radioactivity of the daughter radioisotope;
c) a generator line that permits fluid communication between the reservoir to the generator; and
d) a memory communicatively coupled to the processor when the system is operational, the memory bearing processor-executable instructions when executed on the processor;
   wherein the said radioisotope elution system is able to produce an increased volume of radioactive eluate by initiating two or more consecutive elutions at an increased flow rate and/or increased volume of the eluant, and wherein the shelf-life of the radioisotope generator is detected by measuring the enhancement of signal-to-noise ratio towards the end of a generator life.

Another embodiment of the present invention includes the elution system, wherein the radioisotope generator can be selected from ⁹⁹Mo/^{99m}Tc, ⁹⁰Sr/⁹⁰Y, ⁸²Sr/⁸²Rb, ¹⁸⁸W/¹⁸⁸Re, ⁶⁸Ge/⁶⁸Ga, ⁴²Ar/⁴²K, ⁴⁴Ti/⁴⁴Sc, ⁵²Fe/^{52m}Mn, ⁷²Se/⁷²As, ⁸³Rb/^{83m}Kr, ¹⁰³Pd/^{103m}Rh, ¹⁰⁹Cd/^{109m}Ag, ¹¹³Sn/^{113m}In, ¹¹⁸Te/¹¹⁸Sb, ¹³²Te/¹³²I, ¹³⁷Cs/^{137m}Ba, ¹⁴⁰Ba/¹⁴⁰La, ¹³⁴Ce/¹³⁴La, ¹⁴⁴Ce/¹⁴⁴Pr, ¹⁴⁰Nd/¹⁴⁰Pr, ¹⁶⁶Dy/¹⁶⁶Ho, ¹⁶⁷Tm/^{167m}Er, ¹⁷²Hf/¹⁷²Lu, ¹⁷⁸W/¹⁷⁸Ta, ¹⁹¹Os/^{191m}Ir, ¹⁹⁴Os/¹⁹⁴Ir, ²²⁶Ra/²²²Rn and ²²⁵Ac/²¹³Bi, and thereof.

An embodiment of the present invention includes the radioisotope elution system, wherein the radioisotope generator is ⁸²Sr/⁸²Rb.

An embodiment of the present invention includes the elution system, wherein the ⁸²Sr/⁸²Rb radioisotope generator comprises a semi-automated infusion system.

An embodiment of the present invention includes the elution system, wherein the ⁹⁹Mo/^{99m}Tc radioisotope generator comprises a semi-automated infusion system.

An embodiment of the present invention includes the radioisotope elution system, wherein the detector is selected from the group consisting of positron detector, PMT detector, CZT detector, or combinations thereof.

An embodiment of the present invention includes the radioisotope elution system, wherein the said radioisotope elution system is able to produce an increased amount of radioactive eluate by commencing two or more consecutive elutions at an increased flow rate and/or increased volume of eluant, and wherein the shelf-life of the radioisotope generator is detected by measuring the enhancement of signal-to-noise ratio towards the end of the generator life. An embodiment of the present invention includes the radioisotope elution system, wherein two or more consecutive elutions are able to produce an increased amount of radioactive eluate.

An embodiment of the present invention includes the radioisotope elution system, wherein the amount of radioactive eluate is increased from about 13% to about 120%.

An embodiment of the present invention includes the radioisotope elution system, wherein the back-to-back daily quality control testing is performed in order to identify the purity of the daughter radioisotope.

An embodiment of the present invention includes the radioisotope elution system, wherein the shelf-life of radioisotope generator is measured by the enhancement of coefficient of variation of signal-to-noise ratio and/or contrast-to-noise ratio based on the two or more consecutive elutions.

An embodiment of the present invention includes the radioisotope elution system, wherein the said suitable eluant has a flow rate from about 10 ml/min to about 100 ml/min.

An embodiment of the present invention includes the radioisotope elution system, wherein the radioisotope generator comprises a semi-automated infusion system.

An embodiment of the present invention includes the radioisotope elution system, wherein the radioisotope elution system further comprising a controller and a user interface.

An embodiment of the present invention includes the radioisotope elution system, wherein the controller is characterized in that the controller is configured to display the information of the elution system on the user interface.

An embodiment of the present invention includes the radioisotope elution system, wherein the controller is configured to notify a user of the radioisotope elution system immediately once the available amount of daughter radioisotope reaches a needed amount to infuse through the patient outlet.

An embodiment of the present invention includes the radioisotope elution system, wherein the controller is configured to control the valves of the radioisotope generator.

An embodiment of the present invention includes the radioisotope elution system, wherein the shelf-life of the radioisotope generator is about 60 days.

An embodiment of the present invention includes the radioisotope elution system, wherein the radioisotope generator further comprises one or more valves, a waste container, and a dose calibrator.

An embodiment of the present invention includes a radioisotope elution system comprising:
a) a radioisotope generator to generate a daughter radioisotope eluting from the parent radioisotope by using a suitable eluant;
b) a detector to determine the radioactivity of the daughter radioisotope;
c) a generator line that permits fluid communication between the reservoir to the generator;
d) a memory communicatively coupled to the processor when the system is operational, the memory bearing processor-executable instructions when executed on the processor;
e) a controller; and
f) a user interface;
   wherein the said radioisotope generator is an ⁸²Sr/⁸²Rb generator and the shelf-life of radioisotope generator is detected by measuring the enhancement of signal-to-noise ratio towards the end of a generator life, and wherein the said radioisotope elution system is able to produce an increased amount of radioactive eluate by initiating two or more consecutive elutions at an increased flow rate and/or increased volume of eluant.

Another embodiment of the present invention includes the radioisotope elution system, wherein the threshold is an ⁸²Sr activity less than 0.02 μCi.

An embodiment of the present invention includes a radioisotope elution system comprising:
a) a radioisotope generator to generate a daughter radioisotope eluting from the parent radioisotope by using a suitable eluant;
b) a detector to determine the radioactivity of the daughter radioisotope;
c) a generator line that permits fluid communication between the reservoir to the generator;
d) a memory communicatively coupled to the processor when the system is operational, the memory bearing processor-executable instructions when executed on the processor;
e) a controller; and
f) a user interface;
   wherein the said radioisotope generator can be selected from the group consisting of ⁹⁹Mo/^{99m}Tc, ⁹⁰Sr/⁹⁰Y, ⁸²Sr/⁸²Rb, ¹⁸⁸W/¹⁸⁸Re, ⁶⁸Ge/⁶⁸Ga, ⁴²Ar/⁴²K, ⁴⁴Ti/⁴⁴Sc, ⁵²Fe/^{52m}Mn, ⁷²Se/⁷²As, ⁸³Rb/^{83m}Kr, ¹⁰³Pd/^{103m}Rh, ¹⁰⁹Cd/^{109m}Ag, ¹¹³Sn/^{113m}In, ¹¹⁸Te/¹¹⁸Sb, ¹³²Te/¹³²I, ¹³⁷Cs/^{137m}Ba, ¹⁴⁰Ba/¹⁴⁰La, ¹³⁴Ce/¹³⁴La, ¹⁴⁴Ce/¹⁴⁴Pr, ¹⁴⁰Nd/¹⁴⁰Pr, ¹⁶⁶Dy/¹⁶⁶Ho, ¹⁶⁷Tm/^{167m}Er, ¹⁷²Hf/¹⁷²Lu, ¹⁷⁸W/¹⁷⁸Ta, ¹⁹¹Os/^{191m}Ir, ¹⁹⁴Os/¹⁹⁴Tr, ²²⁶Ra/²²²Rn, and ²²⁵Ac/²¹³Bi generators, and thereof; wherein the shelf-life of radioisotope generator is detected by measuring the enhancement of signal to noise ratio towards the end of a generator life, and wherein the said radioisotope elution system is able to produce an increased volume of radioactive eluate by initiating two or more consecutive elutions at an increased flow rate.

An embodiment of the present invention includes the elution system, wherein the system is further configured to generate an output on a user interface of the determined optimal period of time until initiation of the imaging protocol.

An embodiment of the present invention includes the elution system, wherein the shelf-life of the radioisotope generator is about 60 days.

An another embodiment of the present invention includes an improved process of radioisotope elution system comprising the steps of:
a) generating a daughter radioisotope eluting from the parent radioisotope within a radioisotope generator by using a suitable eluant;
b) determining the radioactivity of the daughter radioisotope by using a detector and a memory communicatively coupled to the processor when the system is operational, the memory bearing processor-executable instructions, when executed on the processor; and
c) initiating two or more consecutive elutions at an increased flow rate and/or increased volume of the eluant in order to increase the amount of radioactive eluate from about 13% to about 120%;
   wherein the shelf-life of the radioisotope generator is detected by measuring the enhancement of coefficient of variation of signal-to-noise ratio and/or contrast-to-noise ratio towards the end of a generator life based on the consecutive elutions, and wherein the extended shelf-life of the said radioactive generator is about 60 days.

An embodiment of the present invention includes a radioisotope elution system comprising the steps of:
a) eluting a daughter radioisotope from the parent radioisotope in a radioisotope generator by using a suitable eluant at a flow rate of from about 10 ml/min to about 100 ml/min;
b) detecting the radioactivity of the daughter radioisotope by using a detector with decay modes of positron emission of 95.5%;
c) permitting the fluid communication of the generator line between the reservoir to the generator; and
d) controlling the memory communicatively coupled to the processor by the controller when the system is operational, the memory bearing processor-executable instructions when executed on the processor;
   wherein the said daughter radioisotope is ⁸²Rb; wherein the said radioisotope elution system is able to produce an increased amount of radioactive eluate from about 13% to about 120% by initiating two or more consecutive elutions at an increased flow rate and/or increased volume of the eluant; and wherein the shelf-life of the radioisotope generator is detected by measuring the enhancement of signal-to-noise ratio towards the end of the generator life. In an embodiment of the present invention includes the principal elements of a radioisotope elution system comprising reservoir of sterile saline solution (e.g. 0.9% Sodium Chloride Injection); a pump for drawing saline from the reservoir through the generator line at the desired flow rate; a generator valve for proportioning the saline flow between a radioisotope generator and a bypass line which circumvents the radioisotope generator; a positron detector located downstream of the merge point at which the generator and bypass flow merge at the fluid line; and a patient valve for controlling the supply of active saline to a patient outlet through the patient fluid line and a waste reservoir. A controller is connected to the pump, positron detector, and valves to control the elution system in accordance with a desired control algorithm and the controller is further connected to a user interface. Additionally, the radioisotope generator and patient valves may be constructed in a variety of ways. In principal, the generator valve may be provided as any suitable valve arrangement capable of proportioning saline flow between the generator and the bypass line. If desired, the generator valve may be integrated with the branch point at which the saline flow is divided. Alternatively, the generator valve may be positioned downstream of the branch point. The said elution system may also have additional elements including any one or more of a printer and USB (Universal Serial Bus; or other communications port) port, a pressure detector, a dose calibrator, a flow regulator, and a UPS (Uninterruptible Power Supply).

The said elution system may be used to assess various aspects of the system, such as a concentration of parent radioisotopes as well as daughter radioisotopes in a fluid that is eluted from the generator, the volume of the fluid that is eluted from the generator, or the pressure of the fluid flowing through at least one portion of the system. Information about these aspects of the system may be gathered by various elements of the system and sent to the controller. The controller and/or user interface computer (which may comprise a processor and memory) may analyze this gathered data to assess the state of the system. The pressure detector is configured to detect the in-line pressure of the bypass line and to convey information about this pressure to the controller. The pressure detector may be configured to detect the in-line pressure elsewhere within the system, such as the feed-line (saline supply-line). The user interface computer is being connected to the said printer, and having a USB port. The said user interface of the user interface computer may be used to generate an output on the user interface that recommends a course of action or no course of action, based on a result of the assessment. The said printer may be used to print out information about the state of the system, such as a concentration of parent radioisotopes as well as daughter radioisotopes in a fluid that is eluted from the generator, the volume of the fluid that is eluted from the generator, the pressure of the fluid flowing through at least one portion of the system, or the information about the shelf life of the generator, which is measured by the enhancement of signal to noise ratio towards the end of the generator life. The USB port may be used to store an indication of the result of the assessment in a memory location, such as a flash drive.

Additionally, the user interface computer may be configured to communicate with a remote computer, such as a server, or a cloud computing service. The user interface computer may upload an indication of the result of the assessment to a computer via a communications network. The remote computer may collect information from multiple computers, and use this collected information to identify the state of a single and/or multiple elution system(s), or aggregate statistics for multiple elution systems and preserve data with security in order to prevent manipulation from any unauthorized users.

An another embodiment of the present invention includes the elution system, wherein the end-life or shelf-life of the radioisotope generator is measured by coefficient of variation of signal-to-noise ratio and/or contrast-to-noise ratio towards the end of the generator life based on two or more consecutive elutions.

Another aspect of the present invention includes the elution system, wherein the end-life or shelf-life of the radioisotope generator is measured by coefficient of variation of signal-to-noise ratio and/or contrast-to-noise ratio towards the end of the generator life based on the back to back daily quality control test of the radioactive eluate.

The controller of the radioisotope generator advantageously prioritize the patient infusions that require the larger/smaller amounts of daughter radioisotope throughout the generator's life, i.e., larger amounts of daughter radioisotope during the beginning of the generator's life (when the available amount in the generator is higher) and lesser amounts of daughter radioisotope during the end of the generator's life (when the available amount in the generator is smaller). The inventors of the present invention found that since there could be an indication for pediatric use, which are in small doses, and there continues to be an increase in the number of solid state/digital cameras being used in clinics. It is possible to identify the smaller doses that may be required for newer cameras and for the pediatric use. These features of the system advantageously support the user with the schedule of the patients so as to maximize the performance of the system and the schedule. The present invention includes that the process/method of the said elution system is configured with two software options, wherein the elution system can indicate automatically the limit, particularly needed when the generator has less activity and/or alternatively, has a dynamic ability to calculate the signal-to-noise ratio and send an alarm to the user and perform the back-to-back elutions in order to discriminate desired radionuclide from undesired (impurities) and clear the generator for use. The present invention further includes that the controller is configured to display a plan for optimizing the number of patients that will be infused during the same day, during the week, during the month or during the entire life of the generator. The present invention includes that the generator life is about 60 days. The plan preferably proposes a specific order for infusing the patients. The plan displayed by the controller, is specific for a particular day and is based on at least one of the following parameters; the quality control result.

The elution system of the present invention further includes a sensor that may be used to sense an amount eluate that has been dispensed from the generator, sense and/or alert the user to discriminate the desired and undesired radioisotopes for detection of breakthrough, which will help in identifying the end-life of the generator. The elution system of the present invention further includes a signaling device that is communicatively connected to the sensor. The term “communicatively connected” or the like herein refers to a relationship of the first and second components characterized in that at least an electrical signal can be conveyed at least from one of the components to the other. The signal and/or alarm can be visible and/or audible, which is generated automatically in response to detecting a desired amount of eluate to be dispensed and/or administered to the patient. The signal and/or alarm is electronically triggered as a result of the breakthrough and/or a result of the amount of eluate that is determined. The signal and/or alarm is at least one of the visually and/or audibly perceptible exteriors of the elution system. The elution system of the present invention further includes a timer to countdown the elapsed time between two elutions. The timer can be reset in the user interface by the user.

The present inventors unexpectedly found that the radioisotope generator provides consistent signal-to-noise and/or contrast-to-noise ratios towards the end-life or shelf-life of the radioisotope generator. The shelf-life of the said radioisotope generator is measured by the enhancement of coefficient of variation of signal-to-noise ratio and/or contrast-to-noise ratio based on the consecutive elutions; and wherein the extended shelf-life of the said radioactive generator is about 60 days. Sr-breakthrough in an Rb-82 eluate is calculated using the equation below:

$${Sr}_{Breakthrough} = \frac{\frac{{Sr}_{collected in eluate}}{{Rb}_{Activity in eluate}}}{0.02} \times 100$$

Thus, all combinations of the various elements described herein are within the scope of the invention.

This invention will be better understood by reference to the experimental data which follow, but those skilled in the art will readily appreciate that the specific experiments detailed are only illustrative of the invention as described more fully in the claims which follow thereafter.

### EXPERIMENTS

A 100mCi (3700 MBq) generator with 60 days shelf-life will provide an Rb-82 eluate of 100mCi (3700 MBq) at calibration date (day 0) and 19.4 mCi (718 MBq) at expiry (day 60). The amount of Sr-82 corresponding to a 50% breakthrough (as per USP limit) in the eluate at each of the two elution time points is very different as summarized in the table 1 below:

TABLE 1

| | Rb-82 eluate | Sr-82 eluate | | Sr- |
|---|---|---|---|---|
| Generator | activity | activity | Sr-82/ | breakthrough |
| age | (mCi) | (μCi) | Rb-82 | (%) |
| Day 0 | 100.0 | 1.0 | 0.01 | 50 |
| Day 60 | 19.4 | 0.2 | 0.01 | 50 |

At day 60 the amount of Sr-82 is much closer to a dose calibrator detection limit for Rb-82 (Sr-82), which roughly can be found around 0.1 μCi. This decreases the accuracy of the measurement.

Solving the Sr-breakthrough equation for the Strontium collected in the eluate indicates that the only way to increase the amount of Sr-82 in the sample is by increasing the amount of Rb-82 collected:

$${Sr}_{collected in eluate} = \frac{{Sr}_{Breakthrough} \times 0.02 \times {Rb}_{Activity in eluate}}{100}$$

The amount of Rb-82 available from the generator depends on the generator age, for close-to-expiry generators the inventors of the present invention perform:
Example 1: Use of a higher flow rate (from about 30 ml/min to about 100 ml/min) during DQC: The amount of Rb-82 collected in the eluate increases by 13%.
Example 2: Two consecutive elutions are collected in the vial (back-to-back elutions). In this case, the amount of Rb-82 is doubled (by 100% increase).
Example 3: Two consecutive elutions at the higher flow rate of about 30 ml/min to about 100 ml/min are collected in the daily quality control (DQC) vial. In this case, the amount of Rb-82 is increased from about 13% to about 120%.
Effect on detection limit and accuracy: All these 3 working examples of the present invention increased the amount of Sr-82 collected for the same breakthrough value as observed in the below-mentioned Table 2. Since these values are farther away from the 0.1 μCi detection limit (higher) of the instrument this leads to improved accuracy of the measurement.

TABLE 2

| | | | | Sr- |
|---|---|---|---|---|
| | | Rb-82 eluate | Sr-82 eluate | break- |
| | Generator | activity | activity | through |
| Scenario | age | (mCi) | (μCi) | (%) |
| Current procedure | Day 60 | 19.4 | 0.19 | 50 |
| Increased flow rate | Day 60 | 21.9 | 0.25 | 50 |
| Back-to-back elution | Day 60 | 38.8 | 0.39 | 50 |
| Increased flow | Day 60 | 43.8 | 0.44 | 50 |
| rate + Back-to-back | | | | |
| elution | | | | |

It is observed that in working example 3, while combining a higher flow rate and a back-to-back elution, the activity of Rb-82 collected is similar to the activity that would be collected from the generator at day 30. This indicates that this approach can also help extend the shelf-life or end-life of the radioisotope generator when the accuracy of the Sr-breakthrough is the limiting factor.

1. A radioisotope elution system comprising:
a) a radioisotope generator to generate a daughter radioisotope eluting from a parent radioisotope by using an eluant;
b) a detector to determine the radioactivity of the daughter radioisotope;
c) a generator line that permits fluid communication between a reservoir to the radioisotope generator; and
d) a memory communicatively coupled to a processor when the radioisotope elution system is operational, the memory bearing processor-executable instructions when executed on a processor;
wherein the radioisotope generator is an ⁸²Sr/⁸²Rb generator and the radioisotope elution system is able to produce an increased amount of the daughter radioisotope by initiating two or more consecutive elutions at an increased flow rate and/or increased volume of the eluant; and
wherein a shelf-life of the radioisotope generator is detected by measuring an enhancement of a signal-to-noise ratio towards the end of a generator life.

2. The radioisotope elution system of claim 1, wherein the detector is selected from the group consisting of positron detector, PMT detector, CZT detector, or combinations thereof.

3. The radioisotope elution system of claim 1, wherein the increased flow rate and/or the increased volume of the eluant is in order to increase the amount of daughter radioisotope.

4. The radioisotope elution system of claim 1, wherein the two or more consecutive elutions are able to produce an increased amount of the daughter radioisotope.

5. The radioisotope elution system of claim 4, wherein the shelf-life of the radioisotope generator is detected by a back-to-back daily quality control testing.

6. The radioisotope elution system of claim 5, wherein the back-to-back daily quality control testing is performed in order to identify the purity of the daughter radioisotope.

7. The radioisotope elution system of claim 1, wherein the shelf-life of the radioisotope generator is detected by measuring the enhancement of the signal-to-noise ratio towards the end of the generator life.

8. The radioisotope elution system of claim 1, wherein the eluant has a flow rate from about 10 ml/min to about 100 ml/min.

9. The radioisotope elution system of claim 1, wherein the radioisotope elution system further comprises a controller and a user interface.

10. The radioisotope elution system of claim 1, wherein the radioisotope generator further comprises one or more valves, a waste container, and a dose calibrator.

11. The radioisotope elution system of claim 9, wherein the controller is characterized in that the controller is configured to display the information of the elution system on the user interface; and wherein the controller is configured to notify a user of the radioisotope elution system immediately once the available amount of daughter radioisotope reaches a needed amount to infuse through the patient outlet.

12. A radioisotope elution system comprising:
a) a radioisotope generator to generate a daughter radioisotope eluting from a parent radioisotope by using an eluant at a flow rate of from about 10 ml/min to about 100 ml/min;
b) a detector to determine the radioactivity of the daughter radioisotope with decay modes of positron emission of 95.5%;
c) a generator line that permits fluid communication between a reservoir to the radioisotope generator; and
d) a memory communicatively coupled to a processor when the system is operational, the memory bearing processor-executable instructions when executed on the processor;
wherein the radioisotope generator is an ⁸²Sr/⁸²Rb generator and the radioisotope elution system is able to produce an increased amount of radioactive eluate from about 13% to about 120% by initiating two or more consecutive elutions at an increased flow rate and/or an increased volume of the eluant; and
wherein a shelf-life of the radioisotope generator is detected by measuring an enhancement of a signal-to-noise ratio towards the end of the radioisotope generator life.

13. The radioisotope elution system of claim 12, wherein the radioisotope elution system further comprises a user interface, one or more valves, a waste container, and a dose calibrator.

14. The radioisotope elution system of claim 1, wherein the radioisotope generator further comprises an automated or semi-automated infusion system.

15. The radioisotope elution system of claim 9, wherein the radioisotope generator further comprises one or more valves and the controller is configured to control the valves.

16. The radioisotope elution system of claim 1, wherein the shelf-life of the radioisotope generator is detected by measuring the threshold of ⁸²Sr activity less than 0.02 μCi.

17. The radioisotope elution system of claim 1, wherein the radioisotope elution system further comprises a sensor that can sense an amount of the daughter radioisotope that has been dispensed from the radioisotope generator.

18. The radioisotope elution system of claim 17, wherein the sensor is configured to provide an alarm that can be visible and/or audible and at least one of the visually and/or audibly perceptible alarms is exterior of the elution system.

19. The radioisotope elution system of claim 17, wherein the sensor automatically alarms upon sensing a limit of low radioactivity and the radioisotope elution system is configured to have a dynamic ability to calculate the signal-to-noise ratio and perform the back-to-back elutions in order to discriminate desired and undesired radioisotopes.

20. A radioisotope elution system comprising:
a) a radioisotope generator to generate a daughter radioisotope eluting from the parent radioisotope by using an eluant in the radioisotope generator;
b) a detector to determine the radioactivity of the daughter radioisotope;
c) a generator line that permits fluid communication between a reservoir to the radioisotope generator;
d) a memory communicatively coupled to a processor when the radioisotope elution system is operational, the memory bearing processor-executable instructions executable on a processor;
e) a controller;
f) a user interface;
g) one or more valves;
h) a waste container; and
i) a dose calibrator;
wherein the radioisotope generator is can be selected from the group consisting of ⁹⁹Mo/^{99m}Tc, ⁹⁰Sr/⁹⁰Y, ⁸²Sr/⁸²Rb, ¹⁸⁸W/¹⁸⁸Re, ⁶⁸Ge/⁶⁸Ga, ⁴²Ar/⁴²K, ⁴⁴Ti/⁴⁴Sc, ⁵²Fe/^{52m}Mn, ⁷²Se/¹²As, ⁸³Rb/^{83m}Kr, ¹⁰³Pd/^{103m}Rh, ¹⁰⁹Cd/^{109m}Ag, ¹¹³Sn/^{113m}In, ¹¹⁸Te/¹¹⁸Sb, ¹³²Te/¹³²I, ¹³⁷Cs/^{137m}Ba, ¹⁴⁰Ba/¹⁴⁰La, ¹³⁴Ce/¹³⁴La, ¹⁴⁴Ce/¹⁴⁴Pr, ¹⁴⁰Nd/¹⁴⁰Pr, ¹⁶⁶Dy/¹⁶⁶Ho, ¹⁶⁷Tm/^{167m}Er, ¹⁷²Hf/¹⁷²Lu, ¹⁷⁸W/¹⁷⁸Ta, ¹⁹¹Os/^{191m}Ir, ¹⁹⁴Os/¹⁹⁴Ir, ²²⁶Ra/²²²Rn, and ²²⁵Ac/²¹³Bi generators,
wherein the radioisotope elution system is able to produce an increased amount of daughter radioisotope by initiating two or more consecutive elutions at an increased flow rate and/or an increased volume of the eluant; and wherein a shelf-life of the radioisotope generator is detected by measuring an enhancement of a signal-to-noise ratio towards the end of the generator life.
